(19) United States
(12) Patent Application Publication
(10) Pub. No.: US 2009/0280100 A1
(43) Pub. Date: Nov. 12, 2009

(54) Preventive or Therapeutic Agent for Inflammatory Ocular-Surface Diseases

(75) Inventors: Junji Yodoi, Kyoto (JP); Hajime Nakamura, Kyoto (JP); Shigeru Kinoshita, Osaka (JP); Chie Sotozono, Kyoto (JP)

Correspondence Address: LAWSON & WEITZEN, LLP, 88 BLACK FALCON AVE, SUITE 345, BOSTON, MA, 02210, US

(73) Assignee: REDOX BIOSCIENCE INC., Kyoto (JP)

(21) Appl. No.: 11/885,020

(22) PCT Filed: Feb. 24, 2006
(86) PCT No.: PCT/JP2006/303468

§ 371 Date: Aug. 24, 2007

(30) Foreign Application Priority Data
Feb. 25, 2005 (JP) 2005-052012

(51) Int. Cl. A61K 38/44; C12N 9/02

(52) U.S. Cl. 424/94.4; 435/189

(57) ABSTRACT

[Problems] The present invention aims to provide a preventive or therapeutic agent for inflammatory ocular-surface diseases.

[Means for Solving Problems] The present invention provides a preventive or therapeutic agent for inflammatory ocular-surface diseases, which comprises polypeptides of thioredoxin superfamily as an active ingredient.

### BACKGROUND OF THE INVENTION

1. Field of Invention

The present invention is related to a preventive or therapeutic agent for inflammatory ocular-surface diseases, and particularly to an agent comprising polypeptide of the thioredoxin superfamily (hereafter referred to as TRXs) as an active ingredient for preventive or therapeutic for inflammatory ocular-surface diseases.

2. Background Art

A commercial drug for the inhibition of inflammatory reactions on the ocular-surface is divided into a steroid and a non-steroid anti-inflammatory drug (NSAID), but both types have serious problems.

A steroid eye drop exhibits a very strong anti-inflammatory effect, but is disadvantageous in that it frequently induces serious adverse effects. As the adverse effects, exacerbation and induction of infectious diseases, delay of wound healing, cataract, glaucoma, and suppression of pituitary-adrenal axis may occur.

It is also greatly disadvantageous in that discontinuation of the drug administration causes a rebound phenomenon in which inflammation is exacerbated worse than the one before the administration (patent literature 1).

A non-steroid anti-inflammatory drug has few adverse effects as various and serious as steroid ant-inflammatory drug, however it is greatly disadvantageous in that its potency is not entirely satisfactory. Moreover, all the non-steroid anti-inflammatory drug is cytotoxic to corneal epithelium. Topical application such as an eye drop, may cause epithelial defect as an adverse effect. The non-steroid drug exhibits an anti-inflammatory effect by inhibiting prostaglandin production.

Since the non-steroid anti-inflammatory drug does not suppress the function of lymphocyte which triggers the induction of inflammatory diseases, it has another big disadvantage in that the administration can be only a symptomatic treatment, but not a radical treatment (patent literature 2).

Because of the above reasons, it has been eagerly awaited that a highly effective preventive or a therapeutic agent with less adverse effects is developed for intractable inflammatory ocular-surface diseases (for example, Stevens-Johnson syndrome).

On the other hand, thioredoxin (TRX) is a 12 kDa multifunctional peptide. It exhibits oxidation-reduction (redox) activity through a disulfide/dithiol exchange reaction between the 2 cysteine residues in its active site sequence: -Cys-Gly-Pro-Cys- (non-patent literature 1). Since TRX was isolated from Escherichia coli as a coenzyme which donates hydrogen ions to ribonucleotide reductase (an essential enzyme for DNA synthesis), thioredoxin has been isolated from and identified in various prokaryotes and eukaryotes.

Adult T-cell leukemia-derived factor (ADF) was initially identified by the present inventors as an IL-2 receptor-derived factor produced by HTLV-1-infected T lymphocytes. ADF is a human thioredoxin.

Intracellular thioredoxin plays an important role in radical scavenging and the control of transcriptional factors related to redox, such as activator protein-1 and nuclear factor-κB (non-patent literature 2).

Human thioredoxin controls the signal transduction of p38 mitogen activating protein kinase (MAPK) and apoptosis signal regulating kinase-1 (ASK-1).

The inventors have reported that TRX is released from a cell to its outside, exhibits cytokine or chemokine actions (non-patent literature 3), and further, extracellular TRX can be transferred into cells (non-patent literature 4).

However, it has not been reported about the relationship between TRXs and inflammatory ocular-surface diseases, and about the inflammation-inhibitory effect of TRXs on the ocular-surface, that is, effects of TRX as a preventive or therapeutic agent for inflammatory ocular-surface diseases.

[Patent literature 1] Japanese laid open application No. 1999-092383
[Patent literature 2] Japanese laid open application No. 1997-295935
[Non-patent literature 1] Redox regulation of cellular activation Ann. Rev. Immunol. 1997; 15:351-369
[Non-patent literature 2] AP-1 transcriptional activity is regulated by a direct association between thioredoxin and Ref-1 PNAS. 1997; 94:3633-3638
[Non-patent literature 3] Circulating thioredoxin suppresses lipopolysaccharide-induced neutrophil chemotaxis PNAS. 2001; 98: 15143-15148
[Non-patent literature 4] Redox-sensing release of thioredoxin from T lymphocytes with negative feedback loops J. Immunol. 2004; 172:442-448

### DISCLOSURE OF INVENTION

### Problems the Invention Aims to Solve

The present invention aims to provide a highly effective preventive or therapeutic agent for inflammatory ocular-surface diseases with less adverse effects.

### Means to Solve Problems

The inventors elucidated a novel action of TRXs, that is, the inflammation-inhibitory effects on the ocular-surface; discovered its effectiveness as a preventive or therapeutic agent for inflammatory ocular-surface diseases, and then have completed the invention.

The invention described in claim 1 relates to a preventive or therapeutic agent for inflammatory ocular-surface diseases comprising polypeptide of thioredoxin superfamily as an effective ingredient.

The invention described in claim 2 relates to the preventive or therapeutic agent of Claim 1, wherein the inflammatory ocular-surface disease is caused by chronic inflammatory oculus or acute inflammatory oculus.

The invention described in claim 3 relates to the preventive or therapeutic agent of Claim 2, wherein the chronic inflammatory oculus suffers from a disease selected from a group consisting of Stevens-Johnson syndrome, ocular pemphigoid, thermal/chemical burn, idiopathic stem cell deficiency, ectodermal dysplasia and intractable keratoconjunctivit epitheliopathy.

The invention described in claim 4 relates to the preventive or therapeutic agent of Claim 2, wherein the acute inflammatory oculus suffers from a disease selected from a group consisting of thermal/chemical burn, corneal infection, penumbra corneal ulcer, Mooren's ulcer, corneal injury, corneal phlyctenule and corneal post-operation.

The invention described in claim 5 relates to the preventive or therapeutic agent according to any of Claims 1 to 4, wherein polypeptide of thioredoxin superfamily is human thioredoxin.

The invention described in claim 6 relates to the preventive or therapeutic agent according to any of Claims 1 to 5, wherein the agent is in a form of eye drop, gel or ointment and contains pharmaceutically acceptable excipient.

### EFFECTS OF THE INVENTION

Since a preventive or therapeutic agent, which comprises polypeptide of the thioredoxin superfamily of the present invention as an active ingredient, strongly inhibits ocular-surface inflammation, it is effective against inflammatory ocular-surface diseases.

Since the active ingredient of the present invention is thioredoxin, which is an endogenous thiol protein expressed in the body, there is no concern regarding adverse effects.

In the present specification, a human thioredoxin (hTRX) refers to a polypeptide consisting of 105 amino acids of Sequence Number 1.

TRXs of the present invention may be molecules of the thioredoxin superfamily other than human thioredoxin, for example, polypeptides with -Cys-Gly-Pro-Cys-, -Cys-Pro-Tyr-Cys-, -Cys-Pro-His-Cys-, or -Cys-Pro-Pro-Cys- in the active center.

The TRX of the present invention is preferably thoredoxin which has -Cys-Gly-Pro-Cys- in the active center, or thioredoxin 2 (mitochondria-specific thioredoxin).

Thioredoxin derivatives of the present invention may be manufactured based on hTRX of Sequence Number 1 by known genetic engineering techniques.

The derivatives may contain one or more amino acids, preferably one or a few amino acids, substituted, deleted, added, or inserted, at amino acids excluding 32 and 35 of Sequence No. 1, preferably excluding 32-35, and the derivatives have an inflammation-inhibitory activity for inflammatory ocular diseases.

Sequence Number 2 presents the nucleic acid sequence of Sequence No. 1.

Application methods of a preventive or therapeutic agent related to the present invention are not specified, and appropriately selected by clinicians.

The preventive or therapeutic agent of the present invention is in the formulation of eye drop, gel, or ointment, and preferably in the formulation of topical application with pharmaceutically acceptable excipients.

Inflammatory ocular-surface diseases, to which the preventive or therapeutic agent of the present invention applied, are not limited to, but may include various inflammatory ocular diseases, for examples, such as infectious ocular diseases (e.g., herpes corneae, bacterial keratitis, bacterial conjunctivitis, fungal keratitis, acanthamoeba keratitis, infectious endophthalmitis, infectious corneal ulcer and the like), corneal injury, post-corneal surgery, cicatrizing keratoconjunctival diseases (e.g., alkali corrosive keratoconjunctivitis, Stevens-Johnson syndrome, ocular pemphigoid and the like), corneal ulcer (e.g., Mooren's ulcer, corneal ulcer that follows chronic rheumatoid arthritis and connective tissue disease, Terrien's corneal degeneration, catarrhal corneal ulcer, infectious corneal ulcer and the like), keratomalacia due to vitamin A deficiency, malacia, neuroparalytic keratitis, diabetic keratopathy, keratoconjunctivitis sicca, keratoconjunctivitis on wearing contact lenses, vernal conjunctivitis, conjunctival allergy, uveitis, Behcet's disease, inflammation after cataract surgery, pseudopterygium and the like.

The agent is particularly useful for the prevention and treatment of keratoconjunctival inflammatory diseases (e.g., Stevens-Johnson syndrome, ocular pemphigoid, thermal/chemical burn, herpes corneae, bacterial keratitis, bacterial conjunctivitis, fungal keratitis, acanthamoeba keratitis, corneal injury, alkali corrosive keratoconjunctivitis, corneal ulcer, keratomalacia due to vitamin A deficiency, neuroparalytic keratitis, diabetic keratopathy, keratoconjunctivitis sicca, keratoconjunctivitis on wearing contact lenses, vernal conjunctivitis, conjunctival allergy and the like).

The agent is also useful for the prevention and treatment of corneal ulcer (including various corneal ulcers described above and those associated with other causes), and particularly infectious corneal ulcer.

It is possible to apply thioredoxin, which is an active ingredient contained in the preventive or therapeutic agent of the present invention, alone or along with generally used carriers.

Examples of the carriers include diluents or excipients, such as binders, disintegrators, surfactants, absorption enhancers, humectants, adsorbents, lubricants, filling materials, extenders, moisturizers, preservatives, stabilizers, emulsifiers, solubilizing agents, osmotic pressure-controlling salts, and buffering agents. These are appropriately selected depending on the dosage unit form of preparations.

If the preventive or therapeutic agent of the present invention is formulated as an eye drop such as liquid, emulsion, and suspending agent, it is preferable that the agent is sterilized and isotonic with tears. In preparing these forms, it is possible to use diluents, for example, such as water, ethyl alcohol, macrogol, propylene glycol, ethoxy isostearyl alcohol, polyoxy isostearyl alcohol, polyoxyethylene sorbitan fatty acid esters and the like.

For the above preparations, an agent of the present invention may contain a sufficient amount of sodium chloride, glucose, or glycerin to prepare isotonic solutions.

Conventional solubilizing agents, buffer, or soothing agents may also be added thereto.

If a preventive or therapeutic agent of the present invention is a liquid preparation, it may be preserved after water is removed by cryopreservation or freeze-drying.

Freeze-dried preparations are dissolved with distilled water for injection at the time of use.

A preventive or therapeutic agent of the present invention may also contain coloring agents, preservatives, or other agents according to needs.

An effective dose of polypeptides of the TRX family can be easily determined by those skilled in the art referring to the conventional techniques. For example, the effective dose is about 0.001 mg-0.1 g at a time, preferably about 0.01-10 mg, and more preferably about 0.1-10 mg.

The dose may be applied once a day or divided into several times a day. It is preferable that the dose is appropriately adjusted depending on the various dosage forms, gender and age of patients, and severity of diseases.

Examples of the present invention are explained by referring to embodiments in more details below, but the invention is not limited to these examples.

### EMBODIMENT 1

40 oculi with ocular-surface diseases were used as subjects. These oculi were classified into 4 groups based on the presence or absence and properties of inflammation: non-inflammatory oculi (7 oculi with neoplastic diseases and the like), oculi with acute inflammation (12 oculis with thermal/chemical burn, infection, and the like), oculi with chronic inflammation (18 oculi with Stevens-Johnson syndrome and the like), and oculi with persistent inflammation after thermal/chemical burn (3 oculi).

For the control, 33 oculi with no ocular diseases were used.

The details are shown in Table 1.

TABLE 1

| | Name of ocular-surface diseases |
|---|---|
| | (sample numbers) |
| Non-inflammatory oculi | Conjunctival squamous cell carcinoma (1), |
| | Conjunctival errosion (1), Recurrent corneal |
| | erosion (1), Corneal perforation (1), Post |
| | corneal surgery (3) |
| Acute inflammatory oculi | Thermal/chemical burn (4), Corneal infection |
| | (6), Penumbra corneal ulcer (1), Corneal |
| | phlyctenosis (1) |
| Chronic inflammatory | Stevens-Johnson syndrome (14), Ectodermal |
| oculi | dysplasia (1), Specific stem cell deficiency |
| | (1), Intractable keratoconjunctival |
| | epitheliopathy accompanied by chronic |
| | persistent inflammation (ocular-surface |
| | disease) (2) |
| Persistent inflammatory | Thermal burn (1), Chemical burn (1), Acute |
| oculi after | exacerbation of ocular pemphigoid (1) |
| thermal/chemical burn, | |
| and the like | |
| Controls | Normal oculi (33) |

### 1. Tear Sampling Method

Tear samples were collected from the inferior lateral tear meniscus under direct slit-lamp observation using 1-μL disposable glass capillary micropipette (Microcaps, Drummond Scientific Co., USA) without topical anesthesia. In order to minimize ocular irritation, care was taken to avoid touching the ocular surface or lid margin, and dim slit lamp light was used. Time spent for collecting tears was less than 1 minutes for each oculi. Minimum amount of tear collected in this study is 1 μL. All collected tears were immediately discharged directly to microcentrifugal tubes using a small pump supplied with capillary micropipettes, centrifuged, and then frozen in −80° C. freezer (Sanyo Co.) until analysis as described below.

### 2. ELISA Analysis

TRX in tears was quantitatively analyzed by ELISA for the above 73 oculi (40 oculi with ocular-surface diseases and 33 control oculi with no disease shown in [Table 1]).

ELISA was performed using a TRX ELISA kit (ELISA kit for Human Thioredoxin, Redox Bioscience Co.), following the attached protocol.

All samples were measured at least 3 times, and the measurements were presented as the means±standard error (SD). The measurements were statistically analyzed using Duncan's test.

The expression level of TRX protein was 171.9±121.3 (ng/ml, mean±SD) in non-inflammatory oculi, 715.2±371.9 (ng/ml, mean±SD) in acute inflammation, 2816.2±1923.1 (ng/ml, mean±SD) in oculi with chronic inflammation, and 5943.1±2520.8 in oculi with persistent inflammation after thermal/chemical burn, and 164.3±104.8 (N=22) in basal and 80.8±51.4 (ng/ml, mean±SD) in reflex tears of normal oculi respectively, showing that the significantly high level of TRX was detected in oculi with chronic and persistent inflammation (Table 2).

TABLE 2

| | TRX concentration (ng/mL) |
|---|---|
| | Mean (ng/mL) ± SD |
| Non-inflammatory oculi | 171.9 ± 121.3 |
| Acute inflammation oculi | 715.2 ± 371.9 |
| Chronic inflammation oculi | 2816.2 ± 1923.1 |
| Persistent inflammation oculi after | 5943.1 ± 2520.8 |
| thermal/chemical burn and the like | |
| Control | 164.3 ± 104.8 |

### 3. Western Blot Analysis

Western blot analysis was performed for the above tear samples with a high level TRX: Nos. 383, 376, 377, and 375 (FIG. 1).

Western blotting was performed by the standard method using 0.5 1 g/mL of anti-human thioredoxin monoclonal antibody (Redox Bioscience Co.).

As shown in FIG. 1, each samples applied were, from the left, TRX protein of 1, 10, and 100 ng/mL, as positive controls, and tear samples of No. 383, No. 376, No. 377, and No. 375.

No. 383 is a tear sample collected from an oculus with ocular pemphigoid. The TRX protein level was 2411.7 ng/mL (4.8 ng of TRX protein in 2 μL of the tear sample) by quantitative ELISA analysis.

No. 376 is a tear sample collected from an oculus after thermal burn. The TRX protein level was 1120.4 ng/mL (4.5 ng of TRX protein in 4 μL of the tear sample) by quantitative ELISA analysis.

No. 377 is a tear sample collected from an oculus with ocular pemphigoid. The TRX protein level was 2221.9 ng/mL (4.4 ng of TRX protein in 2 μL of the tear sample) by quantitative ELISA analysis.

No. 375 is a tear sample collected from an oculus with ocular pemphigoid. The TRX protein level was 7499.6 ng/mL (7.5 ng of TRX protein in 1 μL of the tear sample) by quantitative ELISA analysis.

As a result of the Western blot analysis, TRX bands were clearly confirmed (FIG. 1), and the findings were consistent with the above quantification of ELISA analysis.

### 4. Immunohistological Staining

Immunohistological staining was performed.

The experimental conditions of the immunohistological staining are as follows:

Reaction conditions

| | |
|---|---|
| Fixative: | Zamboni solution 4° C., 5 minutes |
| Washing: | PBS 5 minutes, 3 times |
| Blocking: | 1% BSA in PBS, room temperature, 20 minutes |
| Primary | Anti-human TRX monoclonal antibody (Redox |
| antibody: | Bioscience Co.) was diluted to 1.0 g/mL (3000 times |
| | dilution) with 1% BSA, room temperature, 1 hour |
| Washing: | PBS, 5 minutes, 3 times |
| Secondary | Alexa Fluor 488 goat Anti mouse IgG (Invitogen Corp., |
| antibody: | Carlsbad, CA, USA) was diluted with 1% BSA to 1 |
| | μg/mL (2000 times dilution), room temperature, 1 |
| | hour |
| Washing: | PBS, 5 minutes, 3 times |
| Mounting: | VECTASHIELD with propidium iodide (Vector |
| | Laboratories, Inc., Burlingame, CA, USA) |

FIG. 2 shows stain images of normal-corneal-epithelial-layer stained immunohistologically.

FIG. 3 shows stain images of cicatricial-epithelial-layers covering the cornea stained immunohistologically.

TRX was significantly expressed in the oculus of Stevens-Johnson syndrome, which is a chronic persistent inflammatory oculus, compared to that in the normal conjunctival epithelium (FIG. 3).

In ocular pemphigoid, TRX expression was topically increased particularly in the most superfacel layer of the cicatricial epithelial layer (FIG. 3).

These findings were consistent with the above ELISA result.

FIG. 4 shows stain images of cicatricial-epithelial-layers covering the cornea stained immunohistologically, as in FIG. 3, along with the images showing pathology of the ocular-surface of the corresponding patients.

The expression level of TRX protein was quantitatively determined to be 937.3 ng/mL and 2669.3 ng/mL in the tear samples of the oculi with Stevens-Johnson syndrome and ocular pemphigoid respectively, using ELISA analysis described above.

Based on the above findings, TRX was expressed in the epithelial layer with acute or chronic inflammation of the ocular-surface, suggesting its involvement in the regulation of oxidative stress and inflammation.

### EMBODIMENT 2

Experiments demonstrating “the effect of TRX on cultured human corneal-epithelial-cells” and “the inhibitory effect of inflammation by TRX” are shown below.

### 1. Culture Cell

### Cells Used:

(a) SV-40 immortalized human corneal epithelial cells
(b) Cultured human corneal epithelial cells (purchased from Kurabo Industries Ltd.)
(c) Cultured human corneal epithelial cells (cells which are cultured and propagated from a cornea (purchased from American Eye Bank)).

Cultured cells of (a)-(c) were cultured in 25 cm² T flasks where 10 ml of RPMI culture medium was pored into. The medium contains 15% inactivated fetal bovine serum, 200 mM L-glutamine, 50 mM β-mercaptoethanol, and 100 pM rmIL-3.

After 1 week, the cells were transferred into a middle-size flask, combined with 30 mL of the medium, and subsequently subcultured into middle-size flasks in the next week.

2. Expression of Inflammatory Cytokines (refer to FIG. 5)

Inflammatory cytokines were investigated according to the experimental procedure below.

1) The medium was replaced with that containing 10% or no serum, and 600,000 cells were inoculated in each well of 12-well plates.
2) The medium was replaced with PBS on the following morning, and the cells were irradiated with 50 mJ ultraviolet light (UV).

That is, acute inflammation was induced in cultured cells of (a)-(c) by UV irradiation.

3) Immediately after the irradiation, 1000 μg/mL TRX was added.
4) RNA was collected after 4 hours, and mRNA expressions of IL-6/β-actin and IL-8/β-actin were measured by real-time PCR.

Significantly increased expression of IL-6/β-actin and IL-8/β-actin mRNA was observed 4 hours after the UV irradiation, but the increase was suppressed in the TRX treated group (FIGS. 5 (A) and (B)).

In the corneal epithelial cells, the expression of inflammatory cytokines, IL-6 and IL-8, was increased after the UV irradiation. This increase in the expression is considered to be associated with acute corneal epithelial diseases after the exposure to UV (refer to Kennedy M, Kim K H, Harten B, Brown 3, Planck S, Meshul C, Edelhauser H, Rosenbaum J T, Armstrong Calif., Ansel J C. Ultraviolet irradiation induces the production of multiple cytokines by human corneal cells. Invest Opthalmol V is Sci. 1997 November; 38 (12):2483-91).

From the above experiment, the expression-increasing of the IL-6/β-actin and IL-8/β-actin induced by the UV irradiation was inhibited by the addition of TRX, demonstrating that TRX inhibited the inflammation of the ocular-surface.

3. TUNEL Staining (Investigation of Apoptosis) (refer to FIG. 6)

Apoptosis was investigated by TUNEL staining according to the experimental procedures below.

1) The medium was replaced with that containing 10% or no serum, and cells were inoculated in the medium in a slide chamber.
2) The medium was replaced with PBS on the following day, and the cells were irradiated with 50 ml UV.

That is, acute inflammation was induced in cultured cells of (a)-(c) by UV irradiation.

3) Immediately after the UV irradiation, 1000 μg/mL TRX was added.
4) The cells were fixed in glutaraldehyde 8 hours after the UV irradiation, and subjected to TUNEL staining.

No TUNEL-positive cells were detected in the non-irradiated group. In contrast, apoptosis occurred in epithelial cells 8 hours after the UV irradiation, and many cells were stained TUNEL positive.

In the TRX treated group (50 ml TRX (+)), only a few cells were stained TUNEL positive.

When TUNEL staining was performed in the youngest cells, TRX completely inhibited apoptosis at 1000 g/mL.

UV irradiation induced the apoptosis of corneal epithelial cells, but TRX suppressed the UV-induced apoptosis.

Based on these findings, it was demonstrated that TRX suppressed apoptosis.

The experiment, with three types of the cultured cell (human corneal epithelial cells of (a)-(c) above) resulted in similar findings irrespective to the cell types and media used.

Therefore, it was demonstrated that the preventive or therapeutic agent of the present invention, which comprises polypeptide of the thioredoxin superfamily as an active ingredient, strongly suppresses ocular-surface inflammation, and is effective against inflammatory ocular-surface diseases.

### BRIEF DESCRIPTION OF DRAWINGS

FIG. 1 A result of Western blot analysis is shown. Tear samples having high level of TRX detected are presented.

FIG. 2 Stain images of immunohistologically stained normal corneal epithelial layer are presented.

FIG. 3 Images resulting from the immunohistological staining are presented. The tear samples having high levels of TRX detected were used.

FIG. 4 Images resulting from the immunohistological staining are presented. The tear samples having high levels of TRX detected were used.

FIG. 5 Results of real-time PCR are presented. It is shown that the increased expression of IL-6/β-actin and IL-8/β-actin is suppressed by addition of TRX.

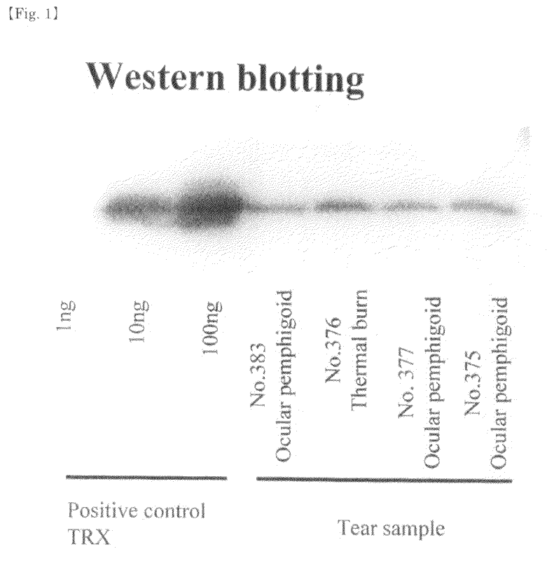

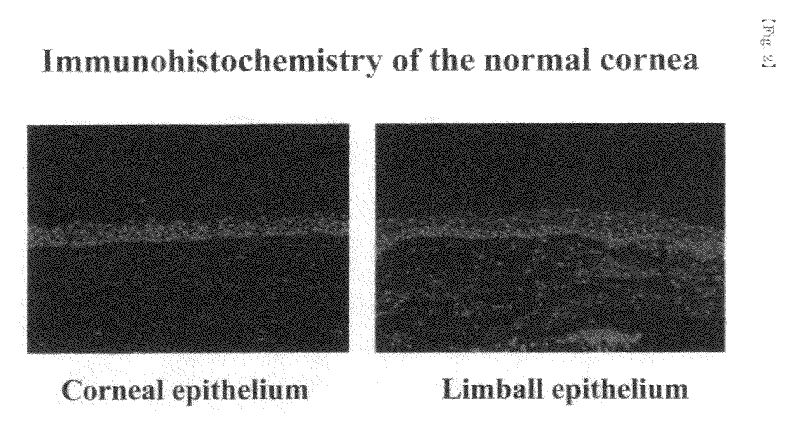

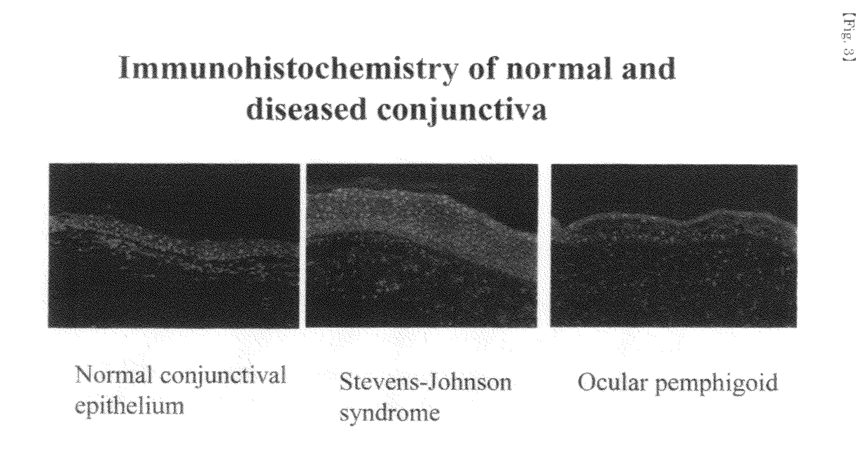

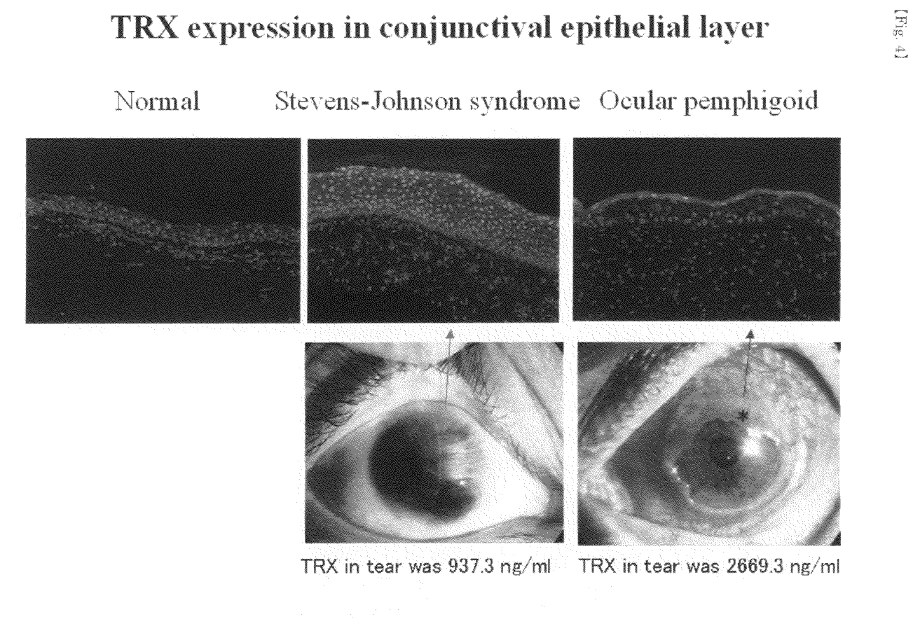

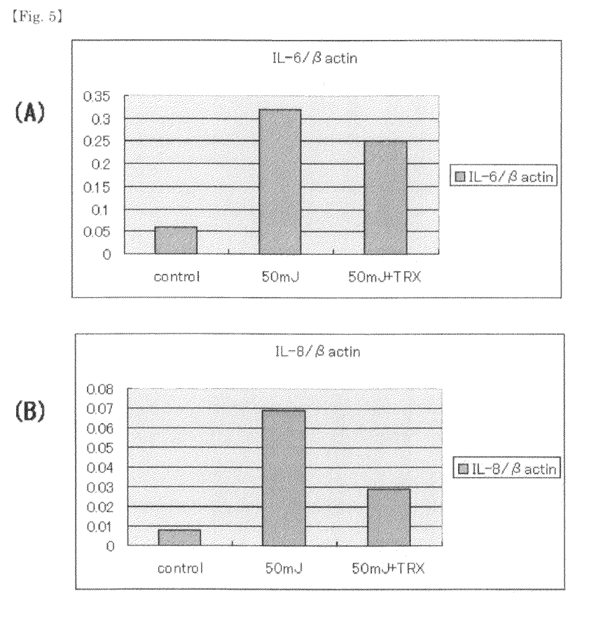

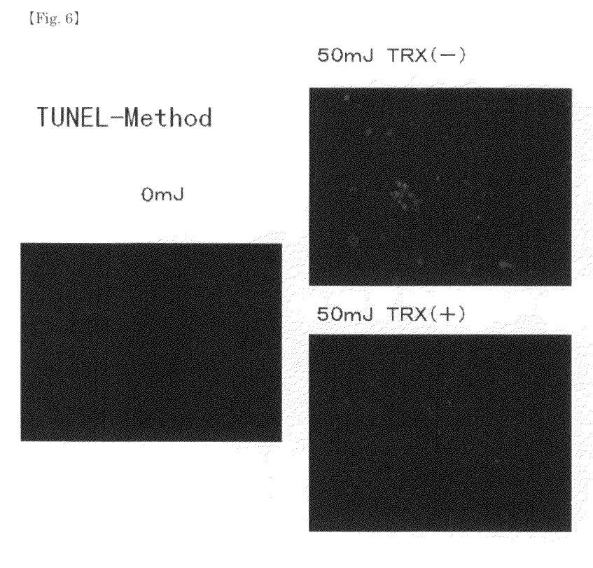

FIG. 6 Images resulting from the TUNEL staining are presented. It is shown that the apoptosis is suppressed by TRX.
Table of Sequences

1. A preventive or therapeutic agent for inflammatory ocular-surface diseases comprising a polypeptide of thioredoxin superfamily as an effective ingredient.

2. The preventive or therapeutic agent of claim 1, wherein the inflammatory ocular-surface disease is caused by chronic inflammatory oculus or acute inflammatory oculus.

3. The preventive or therapeutic agent of claim 2, wherein the chronic inflammatory oculus suffers from a disease selected from a group consisting of Stevens-Johnson syndrome, ocular pemphigoid, thermal/chemical burn, idiopathic stem cell deficiency, ectodermal dysplasia and intractable keratoconjunctivit epitheliopathy.

4. The preventive or therapeutic agent of claim 2, wherein the acute inflammatory oculus suffers from a disease selected from a group consisting of thermal/chemical burn, corneal infection, penumbra corneal ulcer, Mooren's ulcer, corneal injury, corneal phlyctenule and corneal post-operation.

5. The preventive or therapeutic agent of claim 1, wherein the polypeptide of thioredoxin superfamily is human thioredoxin.

6. The preventive or therapeutic agent according to claim 1, wherein the agent is in a form of eye drop, gel or ointment and contains pharmaceutically acceptable excipient.

7. The preventive or therapeutic agent of claim 2 wherein the polypeptide of thioredoxin superfamily is human thioredoxin.

8. The preventive or therapeutic agent of claim 3, wherein the polypeptide of thioredoxin superfamily is human thioredoxin.

9. The preventive or therapeutic agent of claim 4, wherein the polypeptide of thioredoxin superfamily is human thioredoxin.

10. The preventive or therapeutic agent of claim 2, wherein the agent is in a form of eye drop, gel or ointment and contains pharmaceutically acceptable excipient.

11. The preventive or therapeutic agent of claim 3, wherein the agent is in a form of eye drop, gel or ointment and contains pharmaceutically acceptable excipient.

12. The preventive or therapeutic agent of claim 4, wherein the agent is in a form of eye drop, gel or ointment and contains pharmaceutically acceptable excipient.

13. The preventive or therapeutic agent of claim 5, wherein the agent is in a form of eye drop, gel or ointment and contains pharmaceutically acceptable excipient.

14. A preventive or therapeutic agent for inflammatory ocular-surface diseases comprising a polypeptide of thioredoxin superfamily as an effective ingredient, the polypeptide comprising at least one of the sequences selected from the group of -Cys-Gly-Pro-Cys- (SEQ ID NO:3), -Cys-Pro-Tyr-Cys- (SEQ ID NO:4), -Cys-Pro-His-Cys- (SEQ ID NO:5), or -Cys-Pro-Pro-Cys- (SEQ ID NO:6) in the active center of the amino acid sequence of the thioredoxin protein or a derivative thereof having inflammation-inhibitory activity for inflammatory ocular diseases.

15. The preventive or therapeutic agent of claim 14, wherein the derivative comprises one or more amino acids that are substituted, deleted, added, or inserted at a position in the amino acid sequence, wherein the one or more amino acids exclude sequences in the active center comprising amino acid sequences -Cys-Gly-Pro-Cys-(SEQ ID NO:3), -Cys-Pro-Tyr-Cys- (SEQ ID NO:4), -Cys-Pro-His-Cys- (SEQ ID NO:5), or -Cys-Pro-Pro-Cys- (SEQ ID NO:6).


SEQUENCE LISTING (6 sequences)

SEQ ID NO: 1 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 2 - DNA, 318 bp, Homo sapiens
atggtgaagcagatcgagagcaagactgcttttcaggaagccttggacgctgcaggtgataaacttgtagtagttgacttctcagccacgtggtgtgggccttgcaaaatgatcaagcctttctttcattccctctctgaaaagtattccaacgtgatattccttgaagtagatgtggatgactgtcaggatgttgcttcagagtgtgaagtcaaatgcatgccaacattccagttttttaagaagggacaaaaggtgggtgaattttctggagccaataaggaaaagcttgaagccaccattaatgaattagtctaa

SEQ ID NO: 3 - protein, 4 aa, Artificial
  Molecule of thioredoxin superfamily
CGPC

SEQ ID NO: 4 - protein, 4 aa, Artificial
  Molecule of thioredoxin superfamily
CPYC

SEQ ID NO: 5 - protein, 4 aa, Artificial
  Molecule of thioredoxin superfamily
CPHC

SEQ ID NO: 6 - protein, 4 aa, Artificial
  Molecule of thioredoxin superfamily
CPPC